(19) United States
(12) Patent Application Publication
(10) Pub. No.: US 2026/0191904 A1
(43) Pub. Date: Jul. 9, 2026

(54) EXOGENOUS MONOCYTE EXTRACELLULAR VESICLES AND METHOD FOR TREATING INTRACEREBRAL HAEMORRHAGE

(71) Applicants: INSTITUT NATIONAL DE LA SANTÉ ET DE LA RECHERCHE MÉDICALE, Paris (FR); UNIVERSITÉ DE CAEN NORMANDIE, Caen (FR); CENTRE HOSPITALIER UNIVERSITAIRE DE CAEN NORMANDIE, Caen (FR)

(72) Inventors: Sara MARTINEZ DE LIZARRONDO, Caen (FR); Maxime GAUBERTI, Caen Cedex (FR); Fanny POTZEHA, Caen Cedex (FR); Denis VIVIEN, Caen Cedex (FR)

(21) Appl. No.: 19/133,425

(22) PCT Filed: Dec. 5, 2022
(86) PCT No.: PCT/IB2022/000712

§ 371 Date: May 28, 2025

(51) Int. Cl. A61K 35/17; A61K 31/122; A61K 31/196; A61K 31/727; A61K 38/17; A61P 7/04

(52) U.S. Cl. CPC A61K 35/17; A61K 31/122; A61K 31/196; A61K 31/727; A61K 38/17; A61P 7/04

(57) ABSTRACT

Intracerebral hemorrhage (ICH), defined as spontaneous bleeding into the brain, is the deadliest, most disabling, and least treatable form of stroke. The aim of the present invention is to generate large amount of hemostatic mEVs to be used as hemostatic patches in different preclinical models of ICH. Indeed, the exogenous mEVs pf the present invention bearing TF and PSGL-1 improve outcome after collagenase-induced ICH by acting as intravascular hemostatic patches. The present invention thus relates to monocyte extracellular vesicles (mEVs) functionalized with tissue factor (TF) and P-Selectin Glycoprotein Ligand 1 (PSGL-1).

### FIELD OF THE INVENTION

The invention relates to a exogenous monocyte extracellular vesicles and method for the treatment of intracerebral haemorrhage in a subject in need therefore comprising administering to the subject a therapeutically effective amount of a exogenous monocyte extracellular vesicles.

### BACKGROUND OF THE INVENTION

Intracerebral hemorrhage (ICH), defined as spontaneous bleeding into the brain, is the deadliest, most disabling, and least treatable form of stroke. Compared with ischemic stroke and subarachnoid hemorrhage, victims of ICH suffer higher mortality and are left with more severe deficits¹. In contrast to advances in the acute management of subarachnoid hemorrhage and ischemic stroke, effective therapies for ICH are not available, treatment is primarily supportive, and outcomes remain poor². Blood pressure reduction and osmotherapy are usually given in the acute setting, but the effect of these interventions is unclear³⁻⁶. The role of surgical hematoma evacuation remains controversial as well. Previous studies have demonstrated the feasibility of CT-guided stereotaxic thrombolysis and clot aspiration for small deep hematomas and intraventricular local thrombolytic therapy for hastening the removal of intraventricular hemorrhage⁷⁻⁹ but latest trials of these interventions did not improve the proportion of patients who achieved a long-term outcome after ICH^{8, 10}. In recent years, attention has shifted to early hematoma growth as an important cause of early neurological deterioration after ICH. Historically, bleeding in ICH was thought to be completed within minutes of onset, and neurological deterioration during the first day was attributed to cerebral edema and mass effect around the hemorrhage^{11, 12}. Prospective and retrospective studies indicate that early hematoma growth occurs in one third of ICH patients scanned within 3 hours of onset and is associated with poor outcome¹³⁻¹⁶. The three most consistently identified predictors of poor outcome after ICH are hematoma volume, the presence of intraventricular hemorrhage, and depressed level of consciousness¹⁷⁻¹⁹. Of these, hematoma volume has been identified as the single most powerful predictor of 30-day mortality after ICH²⁰. Given the evidence that ongoing bleeding can occur for several hours after onset, it is plausible that ultra-early hemostatic therapy might minimize hematoma volume and improve outcome²¹. In this paradigm, ultra-early hemostatic therapy for ICH could be used as the counterpart to thrombolytic intervention for acute ischemic stroke.

Clinical trials using untargeted hemostatic therapy, such as recombinant activated Factor VII (rFVIIa) which forms a complex with exposed tissue factor (TF), activating the extrinsic coagulation pathway²², showed that this approach is promising. Results showed that rFVIIa administration promoted efficient hemostasis and reduced hematoma growth. Unfortunately, rFVIIa treatment was associated with risk of systemic coagulation or thromboembolic complications^{23, 24}. Targeted treatments able to selectively promote hemostasis at the site of bleeding are therefore necessary.

Circulating extracellular vesicles (EVs) are small (0.1-1 μm) membrane vesicles originating from many different cells by membrane blebbing after activation, apoptosis, or high shear stress^{25, 26}. EVs, as their parental cells, provide cell surface component and are well known to participate in the coagulation process. In in vitro studies with plasma from healthy individuals, EVs enhance thrombin generation, fibrin clot structure and clot stability²⁷.

Importantly, previous studies have reported that monocyte-derived EVs (mEVs), which are the mean source of blood-borne TF28, are recruited to sites of vascular injury in vivo, accumulate during thrombus formation and locally promote thrombin generation²⁹⁻³¹. Thus, the aim of the present invention is to generate large amount of hemostatic mEVs to be used as hemostatic patches in different preclinical models of ICH.

### SUMMARY OF THE INVENTION

The invention relates to a exogenous monocyte extracellular vesicles and method for the treatment of intracerebral haemorrhage in a subject in need therefore comprising administering to the subject a therapeutically effective amount of a exogenous monocyte extracellular vesicles. In particular, the present invention is defined by the claims.

### DETAILED DESCRIPTION OF THE INVENTION

Intracerebral hemorrhage (ICH) is the most severe stroke subtype. Stopping the ongoing bleeding using pro-hemostatic agents is a promising therapeutic strategy that remains limited by serious side effects such as uncontrolled thrombosis. In the present study, the inventors developed an original therapeutic strategy for ICH based on the administration of nanosized extracellular vesicles (EVs) that can trigger the coagulation cascade specifically at the site of active bleeding. They first generated large amounts of EVs from TNF-stimulated THP-1 monocytes in bioreactors. Those monocyte-EVs presented a mean size of ~300 nm, a high pro-hemostatic activity reducing the clotting time in a dose- and TF-dependent manner and a high expression of a targeting protein on their surface (P-Selectin Glycoprotein Ligand 1, PSGL-1). In preclinical models of ICH in mice, intravenous injection of mEVs improved stroke outcome in a dose-dependent manner. mEVs at 1 mg/kg prevented hematoma growth by 43% and improved neurological score at 24 h compared to control mice (p<0.01, n=15/group). These effects were also present in more severe models of ICH (enoxaparin or warfarin treated mice). Importantly, the beneficial effect was blocked when using antibodies blocking either TF or PSGL-1, suggesting that both the pro-coagulant activity and the ability to target damaged brain vessels are mandatory for the therapeutic efficacy of EVs. To conclude, exogenous mEVs bearing TF and PSGL-1 improve outcome after collagenase-induced ICH by acting as intravascular hemostatic patches.

### The Extracellular Vesicle (EV)

The present invention relates to a monocyte derived extracellular vesicle (mEV) functionalized with tissue factor (TF) and P-Selectin Glycoprotein Ligand 1 (PSGL-1).

As used herein, the term “extracellular vesicle” or “EV” has its general meaning in the art and is a collective term for different types of membrane-surrounded structures with overlapping composition, density, and sizes (ranging from 30 to >1,000 nm in diameter). In accordance with the recommendations of the International Society for Extracellular Vesicles (ISEV), the term includes but is not limited to exosomes, ectosomes, microvesicles particles apoptotic bodies, argosomes, blebbing vesicules, budding vesicules, dexosomes, ectosomes, exosomes-like vesicules, exosomes, exovesicules, extracellular membrane vesicules, matrix vesicules, membrane particules, membrane vesicules, microparticules, microvesicles, nanovesicles, oncosomes, prominosomes, prostasomes, shedding microvesicles, shedding vesicles, and tolerosomes. Up to now EV are considered to be secreted by all cell types and are present in high amount in all biological fluids analyzed so far (serum, urine/plasma, saliva, cerebrospinal fluid . . . ). EV may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.).

As used herein, the term “functionalized” refers to the fact that the EV of the present invention incorporates in its membrane a polypeptide of interest (e.g. the ERV syncytin of the present invention).

As used herein, the terms “isolated” “isolating” “purified” “purifying,” “enriched,” and “enriching,” as used herein with respect to cells, means that the EVs at some point in time were separated, purified, and capable of therapeutic use. “Highly purified,” “highly enriched,” and “highly isolated,” when used with respect to said extracellular vesicles, indicates that the cells of interest are at least about 70%, about 75%, about 80%, about 85% about 90% or more of the cells, about 95%, at least 99% pure, at least 99.5% pure, or at least 99.9% pure or more of the cells, and can preferably be about 95% or more of the EVs.

As used herein, the term “donor cell” means a cell that is suitable for the production of the EVs of the present invention.

As used herein, the term “target cell” means a cell with which fusion with a EV of the present invention is desired.

As used herein, the term “monocyte” has its general meaning and relates to a type of leukocyte or white blood cell. They are the largest type of leukocyte in blood and can differentiate into macrophages and conventional dendritic cells. As a part of the vertebrate innate immune system monocytes also influence adaptive immune responses and exert tissue repair functions.

As used herein, the term “monocyte-EVs” (mEVs) relates to a monocyte derived extracellular vesicle.

In a particular embodiment the monocyte-EVs is an exogenous monocyte extracellular vesicles.

In a particular embodiment the monocyte-EVs of the present invention have a diameter of about 300 nm.

As used herein, the term “modified” or “engineered” relative to naturally-occurring cell-derived vesicles, refers to cell-derived vesicles (e.g., extracellular vesicles such as monocyte) that have been altered such that they differ from a naturally occurring cell-derived vesicles

In some embodiment, the monocyte-EVs of the present invention is bearing P-Selectin Glycoprotein Ligand 1 allowing the targeting.

As used herein, the term “P-Selectin Glycoprotein Ligand 1” (PSGL-1), also known as SELPLG or CD162 (cluster of differentiation 162), is a glycoprotein found on white blood cells and endothelial cells that binds to P-selectin (P stands for platelet), which is one of a family of selectins that includes E-selectin (endothelial) and L-selectin (leukocyte). Selectins are part of the broader family of cell adhesion molecules. PSGL-1 can bind to all three members of the family but binds best (with the highest affinity) to P-selectin. PSGL-1 has the following Gene ID: 6404 and the following human UniProt number: Q14242.

As used herein, the term “biological activity of PSGL-1” refers to the capacity of targeting of this protein PSGL-1.

In some embodiment, the monocyte-EVs of the present invention is bearing a tissue factor having a coagulant effect and activity.

As used herein, the term “tissue factor” (TF) also called platelet tissue factor, factor III, or CD142, is a protein encoded by the F3 gene, present in subendothelial tissue and leukocytes. Its role in the clotting process is the initiation of thrombin formation from the zymogen prothrombin. Tissue factor belongs to the cytokine receptor protein superfamily and consists of three domains: 1) an extracellular domain, which consists of two fibronectin type III modules whose hydrophobic cores merge in the domain-domain interface. This serves as a (probably rigid) template for factor VIIa binding. 2) a transmembrane domain and 3) a cytosolic domain of 21 amino acids length inside the cell which is involved in the signaling function of TF. TF has has the following Gene ID: 2152 and the following human UniProt number: P13726.

As used herein, the term “biological activity of tissue factor” refers to the coagulant effect of this protein TF.

In some embodiment, the monocyte-EVs of the present invention bearing TF and a PSGL-1 is having a highest level of procoagulant activity compare to an monocyte-EVs not bearing TF and a PSGL-1.

As used herein, the term “highest” relates to a great, or greater than normal, in quantity, size, or intensity.

In some embodiment, the concentration of PSGL-1 is measured.

As used herein, the term “concentration of PSGL-1” relates to a concentration of PSGL-1 at the surface of the EVs of the present invention.

In some embodiment, the monocyte-EVs presented a high concentration of PSGL-1 on their surface.

In a particular embodiment, the concentration of PSGL-1 (i.e. the high concentration of PSGL-1) is superior to 0.01 ng PSGL-1/μg monocyte-EVs proteins. Preferably, the concentration of PSGL-1 (i.e. the high concentration of PSGL-1) is superior to 0.1 ng PSGL-1/μg monocyte-EVs proteins. More preferably, the high concentration of PSGL-1 is superior to 0.2 ng PSGL-1/μg monocyte-EVs proteins.

Test for determining the high concentration of PSGL-1 are well known to the person skilled in the art. In a preferred embodiment, PSGL-1 high concentration may be measured by ELISA after extraction of the proteins from the monocyte-EVs.

As used herein, the term “Enzyme-linked immunosorbent assay” (ELISA) has its general meaning in the art and refers to an assay which uses a solid-phase type of enzyme immunoassay (EIA) to detect the presence of a ligand (commonly a protein) in a liquid sample using antibodies directed against the protein to be measured. More particularly, an ELISA method is used, wherein the wells of a microtiter plate are coated with a set of antibodies or a fragment thereof. A biological sample containing or suspected of containing PSGL-1 or a fragment thereof is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

As used herein, the term “targeting moiety” refers to any molecule that binds specifically to a target (e.g. to PSGL-1 and TF).

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (1) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the antibodies described hereafter, the CDRs have been determined using CDR finding algorithms from www.bioinf.org.uk—see the section entitled «How to identify the CDRs by looking at a sequence» within the Antibodies pages.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “single domain antibody”, “sdAb” or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference.

One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

### Method of Treating Intracerebral Hemorrhage

As used herein, the term “subject” refers to a mammal, such as a rodent, a feline, a canine, and a primate. Particularly, the subject according to the invention is a human. Particularly, the subject according to the invention is an adult. Particularly, the subject according to the invention is a child. Particularly, the subject according to the invention is a teenager. Particularly, the subject according to the invention is a new born. As used herein, the term “subject” encompasses “patient”.

As used herein, the term “Intracerebral hemorrhage” also known as cerebral bleed, intraparenchymal bleed, and hemorrhagic stroke, or haemorrhagic stroke, relates to a sudden bleeding into the tissues of the brain, into its ventricles, or into both. It is one kind of bleeding within the skull and one kind of stroke. Symptoms can include headache, one-sided weakness, vomiting, seizures, decreased level of consciousness, and neck stiffness. Often, symptoms get worse over time. Causes include brain trauma, aneurysms, arteriovenous malformations, and brain tumors. The largest risk factors for spontaneous bleeding are high blood pressure and amyloidosis.

In some embodiment, the intracerebral hemorrhage is stroke.

As used herein, the term “stroke” relates to a disease that affects the arteries leading to and within the brain. A stroke occurs when a blood vessel that carries oxygen and nutrients to the brain is either blocked by a clot or bursts (or ruptures). When that happens, part of the brain cannot get the blood (and oxygen) it needs, so it and brain cells die.

In some embodiment, the exogenous mEVs bearing TF and PSGL-1 of the present invention acts as a intravascular hemostatic patches.

As used herein, the term “hemostatic patch” is indicated for use during a bleeding on internal organs as an adjunct to hemostasis for minimal, mild, moderate bleeding sites.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiment, the administration of exogenous extracellular vesicles bearing TF and PSGL-1 will activate coagulation at the site of bleeding, reducing hematoma growth in intracranial haemorrhage.

As used herein the terms “administering” or “administration” refer to the act of injecting or otherwise physically delivering a substance as it exists outside the body (e.g., exogenous mEVs bearing TF and PSGL-1) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In the context of the invention, the subject is administered of exogenous mEVs bearing TF and PSGL-1 according to the invention by intravenous administration.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The present invention relates also to a pharmaceutical composition comprising the exogenous mEVs bearing TF and PSGL-1 as described above. The exogenous mEVs bearing TF and PSGL-1 may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to one or more of the following agents: solvents such as olive oil, olive oil refined, cottonseed oil, sesame oil, sunflower seed oil, peanut oil, wheat germ oil, soybean oil, jojoba oil, evening primrose oil, coconut oil, palm oil, sweet almond oil, aloe oil, apricot kernel oil, avocado oil, borage oil, hemp seed oil, macadamia nut oil, rose hip oil, pecan oil, hazelnut oil, sasanqua oil, rice bran oil, shea butter, corn oil, camellia oil, grape seed oil, canola oil, castor oil, and combinations thereof, preferably olive oil refined, emulsifiers, suspending agents, decomposers, binding agents, excipients, stabilizing agents, chelating agents, diluents, gelling agents, thickening agent such as beeswax and/or petroleum jelly, preservatives, lubricants, absorption delaying agents, liposomes, antioxidants such as butylhydroxytoluene or butylhydroxyanisole, and the like. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Particularly, the pharmaceutical composition is formulated into a topical formulation that can be directly applied to the skin, for example, a skin suffering from skin afflictions. The topical formulation suitable for the pharmaceutical composition may be an emulsion, a gel, an ointment, a cream, a patch, an embrocation, an aerosol, a spray, a lotion, a serum, a paste, a foam, or a drop. In one embodiment of this application, the pharmaceutical composition is formulated into an external preparation by admixing the extract according to this application with a base such as those that are well known and commonly used in the art.

A further object of the present invention relates to a composition that comprises an amount of the monocyte-EVs of the present invention (“monocyte-EV composition”). Compositions as described herein encompass pharmaceutical compositions that are used for the purpose of performing a method of therapy in subject in need thereof, which includes non-human mammals and human individuals in need thereof. Compositions of the invention may be formulated for delivery to animals for veterinary purposes (e.g., livestock such as cattle, pigs, etc), and other non-human mammalian subjects, as well as to human subjects. For instance, the EVs may be formulated with a physiologically acceptable carrier for use in gene transfer and gene therapy applications. In some embodiments, the said composition further comprises one or more transduction helper compounds. The transduction helper compounds are preferably selected in a group comprising cationic polymers, as described notably by Zuris et al. (2015, Nat Biotechnol, Vol. 33 (n°1): 73-80). The transduction helper compound may be selected in a group comprising polybrene (that may be also termed hexadimethrine bromide), protamine sulfate, 12-myristate 13-acetate (also termed phorbol myristate acetate or PMA, as described by Johnston et al., 2014, Gene Ther, Vol. 21 (12): 1008-1020), vectofusin (as described by Fenard et al., 2013, Molecular Therapy Nucleic Acids, Vol. 2: e90), poloxamer P338 (as described by Anastasov et al., 2016, Lentiviral vectors and exosomes as gene and protein delivery tools, in Methods in Molecular Biology, Vol. 1448:49-61), RetroNectin® Reagent (commercialized by Clontech Laboratories Inc.), Viral Plus® transduction enhancer (commercialized by Applied Biological Materials Inc.), TransPlus® Virus Transduction Enhancer (commercialized by Clinisciences), Lentiboost® (commercialized by Sirion Biotech), or ExpressMag® Transduction System (commercialized by Sigma-Aldrich). As shown in the examples herein, the said cationic transduction helper compound may consist of polybrene. The EVs may be formulated in a conventional manner using one or more physiologically acceptable carriers or excipients. The EVs may be formulated for parenteral administration by injection, e.g., by bolus injection or continuous infusion. Formulations for injection may be presented in unit dosage form, e.g., in ampoules or in multi-dose containers, with an added preservative. The EV compositions may take such forms as suspensions, solutions or emulsions in oily or aqueous vehicles, and may contain formulation agents such as suspending, stabilizing and/or dispersing agents. Liquid preparations of the EV compositions may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts. Alternatively, the compositions may be in powder form for constitution with a suitable vehicle, e.g., sterile pyrogen-free water, before use.

The monocytes-EVs compositions of the invention may be administered to a subject at therapeutically effective doses to provide the therapeutic effects. In some embodiments, an amount of monocytes-EVs composition of the invention is administered at a dose unit that is in the range of about 0.1-5 micrograms (μg)/kilogram (kg). To this end, the monocytes-EVs composition of the invention may be formulated in doses in the range of about 7 mg to about 350 mg to treat to treat an average subject of 70 kg in body weight. The amount of monocytes-EVs composition of the invention that may be administered may be selected in a group comprising 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg or 5.0 mg/kg. The dose of EVs in a unit dosage of the composition may be selected in a group comprising 7 mg, 8 mg, 9 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg, especially for treating an average subject of 70 kg in body weight. These doses can be given once or repeatedly, such as daily, every other day, weekly, biweekly, or monthly. In some embodiments, the composition may be administered to a subject in one dose, or in two doses, or in three doses, or in four doses, or in five doses, or in six doses or more. The interval between dosages may be determined based the practitioner's determination that there is a need thereof.

The monocytes-EVs compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In some embodiments, the composition may be in liquid or solid (e.g. lyophilized) form.

In some embodiment, the exogenous mEVs bearing TF and PSGL-1 of the present invention is administered to the subject in combination with another active ingredient.

In a particular embodiment, the exogenous mEVs bearing TF and PSGL-1 of the present invention is administered to the subject in combination with fibrinolysis inhibitors, such as tranexamic acid or antagonists of anticoagulant treatments, such as protamine for heparin, specific inhibitors for direct oral anticoagulants (e.g. andexanet-alpha/idarucizumab), coagulation factors, such as prothrombin complex concentrate or vitamin K for anti-vitamin-K.

Production of the Monocyte-Derived Extracellular Vesicles (mEVs)

The present invention also relates to a method for obtaining the monocyte-derived extracellular vesicles of the present invention.

In some embodiment, the mEVs of the present invention are produced in bioreactors after pro-inflammatory cytokine stimulation or shear stress.

As used herein, the term “cytokines” relates to small secreted proteins released by cells which have a specific effect on the interactions and communications between cells. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). There are both pro-inflammatory cytokines and anti-inflammatory cytokines.

As used herein, the term “pro-inflammatory cytokine” are produced predominantly by activated macrophages and are involved in the up-regulation of inflammatory reactions. There is abundant evidence that certain pro-inflammatory cytokines such as IL-1β, IL-6, and TNF-α are involved in the process of pathological pain.

In some embodiment, the mEVs of the present invention are produced in bioreactors after pro-inflammatory cytokine stimulation.

In a particular embodiment, the human monocytic cell-line THP-1 were grown in bioreactors and treated with Tumor Necrosis Factor (TNF) to generate TF+ mEVs. Isolated mEVs were finely characterized by number, size and surface antigens (TF, CD14 and P-Selecting Ligand1) by laser-scanning confocal microscopy and flow cytometry.

As used herein, the term “shear stress” relates to a type of stress that acts coplanar with cross section of material. Shear stress arises due to shear forces. They are the pair of forces acting on opposite sides of a body with the same magnitude and opposite direction. Shear stress is a vector quantity.

As used herein, the term “flow cytometry” has its general meaning in the art and relates to a technology that provides rapid multi-parametric analysis of single cells in solution. Flow cytometers utilize lasers as light sources to produce both scattered and fluorescent light signals that are read by detectors such as photodiodes or photomultiplier tubes.

In some embodiments, the EVs of the present invention are prepared by any method well known in the art. In some embodiments, the EVs of the present invention are prepared by methods for 3D culture that are well known in the art, and include, but are not limited to standard culture in 2D flasks, hanging drop culture, culturing on matrices, culturing on microcarriers, culturing on synthetic extracellular scaffolds, culturing on chitosan membranes, culturing under magnetic levitation, suspension culture in rotating bioreactors, or culturing under non-contact inhibition conditions. See, e.g., Haycock J W. (2011). “3D cell culture: a review of current approaches and techniques.”. Methods Mol Biol. 695:1-15; Lee, J; Cuddihy M J, Kotov N A. (14 Mar. 2008). Three-dimensional cell culture matrices: state of the art. doi: 10.1089/teb.2007.0150; Pampaloni, Francesco (October 2007). “The third dimension bridges the gap between cell culture and live tissue”. Nature Reviews 8:839-845; and Souza, Glauco (14 Mar. 2010). “Three-dimensional tissue culture based on magnetic cell levitation”. Nature Nanotechnology: 291-296; the entire content of each are hereby incorporated by reference.

In some embodiment, the present invention relates to the generation of a large amounts of EVs from TNF-stimulated THP-1 monocytes in bioreactors. Those monocyte-EVs presented a mean size of ~300 nm, a high pro-hemostatic activity reducing the clotting time in a dose- and TF-dependent manner and a high expression of a targeting protein on their surface (P-Selectin Glycoprotein Ligand 1, PSGL-1).

In some embodiment, the method of production of the monocyte-EVs of the present invention is defined in the EXAMPLE.

The invention will be further illustrated by the following figures and examples. However, these examples and figures should not be interpreted in any way as limiting the scope of the present invention.

### FIGURES

FIG. 1: Hemostatic action of mEVs in a mouse tail bleeding model. (A) Schematic representation of the procedure where EVs were injected (1 mg/kg~4X10E9 EVs, retro-orbital) 1 minute after tail incision (B) Hemostatic efficacy of mEVs administration compared to control (saline, n=5 mice/group) (C) Schematic representation of the procedure where EVs were injected (1 mg/kg, retro-orbital) 1- or 15 minutes before tail incision. (D and E) Hemostatic efficacy of mEVs compared to control (saline, n=5 mice/group). All data are means±SEM; (**P<0.01). ns, not significant.

FIG. 2: mEVs efficiently reduce hematoma growth and clinical outcome in mild-ICH stroke model. Effect of mEVs administration (1 mg/kg~4X10E9 EVs) on hematoma expansion in a mild model of ICH (collagenase intra-striatal injection). (A) Schematic overview of the experimental protocol. (B) Hematoma volume at 24 hours in controls and the treated mice (mEVs, 1 mg/kg) (n=15 per group). (C and D) Clinical Score at 4 hours and 24 hours. (E) Percentage of weight loss (%) in both groups at 0-24 h and 0-72 h. *P<0.05 and *P<0.01 vs control (saline).

FIG. 3: mEVs efficiently reduce hematoma growth and clinical outcome in severe-ICH stroke model. Effect of mEVs administration (1 mg/kg~4X10E9 EVs) on hematoma expansion in a severe model of ICH of anticoagulated mice (enoxaparin treatment and collagenase intra-striatal injection). (A) Schematic overview of the experimental protocol. (B) Hematoma volume at 24 hours in controls and the treated mice (mEVs, 1 mg/kg) (n=15 per group). (C and D) Clinical Score at 4 hours and 24 hours. (E) Percentage of weight loss (%) in both groups at 0-24 h and 0-72 h. **P<0.01 and ***P<0.001 vs control (saline).

FIG. 4: mEVs beneficial effect on hematoma growth and clinical outcome is dependent on TF activation. Effect of mEVs administration (1 mg/kg~4X10E9 EVs) on hematoma expansion in a mild model of ICH (collagenase intra-striatal injection). For this experience mEVs were pre-incubated with either anti-TF blocking antibody or their respective Isotype control. (A) Schematic overview of the experimental protocol. (B) Hematoma volume at 24 hours in control (isotype-EVs) and anti-TF mice (anti-TF-mEVs) (n=20 per group). (C and D) Clinical Score at 4 hours and 24 hours. (E) Percentage of weight loss (%) in both groups at 0-24 h and 0-72 h. **P<0.01 and ***P<0.001 vs control (saline).

FIG. 5: mEVs beneficial effect on hematoma growth and clinical outcome is dependent on PSGL-1 binding. Effect of mEVs administration (1 mg/kg~4X10E9 EVs) on hematoma expansion in a mild model of ICH (collagenase intra-striatal injection). For this experience mEVs were pre-incubated with either anti-PSGL-1 blocking antibody or their respective Isotype control. (A) Schematic overview of the experimental protocol. (B) Hematoma volume at 24 hours in control (isotype-EVs) and anti-PSGL-1mice (anti-PSGL-1-mEVs) (n=15 per group). (C and D) Clinical Score at 4 hours and 24 hours. (E) Percentage of weight loss (%) in both groups at 0-24 h and 0-72 h. **P<0.01 and ***P<0.001 vs control (saline).

FIG. 6: Quantification of human PSGL-1 levels in different EVs populations by ELISA. Monocyte cell-line (THP-1 cells) and human dermal microvascular endothelial cells (HMEC-1) were stimulated with different cytokines (either TNF (1-10 or 100 ng/ml), LPS (10 ng/ml) or PMA (10 ng/mL)) for 48 h in order to promote EVs production. Then, EVs were purified by serial centrifugations, as previously described, lysed with TNT (Tris-NaCl-Triton) buffer to measure total protein with BCA and finally used for human PSGL-1 ELISA analysis (Biotechne). PSGL-1 levels were represented by the concentration of PSGL-1 (in ng) by total EVs protein (in μg). TNF=tumor necrosis factor, PMA=Phorbol-12-myristate-13-acetate, LPS=Lipopolysaccharide, HMEC=Human dermal microvascular endothelial cell-line, mEVs=monocyte-EVs, N.D.=No detection.

### EXAMPLE

### Material & Methods

### Reagents

Tumor Necrosis Factor was purchased from PeproTech (Rocky Hill, NJ). THP-1 cell line (ATCC® TIB-202™), fetal bovine serum, Carboxyfluorescein succinidimyl ester (CFSE) came from Sigma. INTEM-S, EXTEM-S came from ROTEM®. The cell plasma membrane staining kit—Orange Fluorescence Cytopainter was purchased from Abcam (France). The Lymphoprep™, Pennicilin/Streptomycin, mouse monoclonal antibody anti-phosphatidylserine, human monoclonal antibody anti-TF (HTF-1), goat anti-collagen type IV (Col IV) used for immunohistochemical analyses were obtained from Southern Biotech (Birmingham, AL, USA). Donkey anti-goat antibody F (ab)′2 fragments were purchased from Jackson ImmunoResearch (West Grove, PA, USA). The purified mouse anti-human PSGL-1 (CD162, clone KPL-1) and CD-14 were purchased from BD Biosciences™.

### In Vitro THP-1 Monocyte Culture

Human monocyte cell line, THP-1 cells (obtained from ATCC) were cultivated in 0.2 μm filtered Roswell Park Memorial Institute (RPMI) 1640 medium (Sigma Aldrich) supplemented with 10% EVs-depleted Fetal Calf Serum (FCS) and 2% penicillin/streptomycin, at 37° C. with 5% CO₂. 10×10⁶ cells were cultured in T75 Flasks and 0.05 mM of β-Mercaptoethanol was added to reduce cellular aggregates.

THP-1 mEVs Production in Bioreactors after TNF Stimulation

A minimum of 25×10⁶ THP-1 cells were cultured in a cell suspension bioreactor (CeLLine, Wheaton®). Monocytes were placed in the cell compartment on a 10 kDa semi-permeable membrane and a 0.2 μm filtered complete medium (RPMI 1640) containing 20% EVs-depleted FBS and 1% Penicillin-Streptomycin was added in the nutrient compartment, and RPMI 1640+1% Penicillin-Streptomycin in the upper compartment. Cells were kept in the bioreactor for 72 hours, after that time, in order to promote mEVs generation, THP-1 cells were stimulated 100 ng/ml of recombinant human Tumor Necrosis Factor (TNF) (PeproTech, Rocky Hill, NJ) for 48 h, unless otherwise stated. After that, cellular debris of the supernatant was removed by centrifugation at 2000×G for 5 minutes. mEVs were purified after ultracentrifugation at 20 000×G for 90 minutes at 4° C. Purified mEVs were washed twice and resuspended in 0.1 μm filtered NaCl/Hepes buffer (150 mM NaCl and 10 mM HEPES, pH=7.4) and stored at −80° C.

### Protein Measurement

The protein content of mEVs was determined using Pierce BCA protein Assay Kit (Thermo Scientific, Weston, FL, United States). The BCA working reagent and protein standard were prepared by following the instructions provided by the manufacturer. Bovine serum albumin was used as a standard.

### Size

The size distribution was determined using two independent methods. Size was measured at baseline using Laser Scanning Confocal Microscopy method and using the Nanoparticle tracking analysis.

mEVs Immobilization and Immunolabeling Assessment

To immobilize the mEVs, micro-wells (μ-Slides, Ibidi) were first coated overnight with 2 μg/ml of dopamine diluted in water. Purified mEVs, diluted in NaCl/Hepes buffer, were incubated at 37° C. during 30 minutes in the presence of Cell Plasma Membrane Marker (CPMM, Abcam). This protocol allows labelling virtually all cell-derived mEVs (data not shown). Then, mEVs diluted in NaCl/Hepes buffer were then seeded in coated micro-wells and were immobilized in the dark at room temperature in the absence of stirring. Immunofluorescence was performed using monoclonal mouse antibodies against either TF, PSGL-1 or CD-14 (1:1500).

### Laser Scanning Confocal Microscopy

Laser-scanning confocal microscopy (LSCM) was performed using an inverted Leica SP8 confocal microscope (Leica Microsystems SAS) equipped with an Argon Gas laser and an X40 NA=1.4 oil immersion objective. For CPMM detection, excitation was set at 546 nm and emission filters between 560 and 650 nm. Field of view was set at 1 μm×1 μm with a 1024×10²⁴ planar matrix (pixel size=97.6 nm×97.6 nm).

### Image Analysis

An automated segmentation method was developed with the ImageJ software (v1.45r, NIH). CPMM staining was first used to identify mEVs and estimate their number and size using automatic Otsu-thresholding and the integrated “particle analysis” setting. CPMM positive particles were estimated using the “ROI manager” of ImageJ. In particular, the Feret's diameters of the MPs were computed.

### Western Blot

Lysed mEVs samples were resolved for the western blot on 8% SDS-Page gels (Biorad) gels in reduced (for TF) and non-reduced (for PSGL-1) conditions. Proteins were transferred onto a polyvinylidene difluoride (PVDF) membrane by Fast Blot transfer stacks (BioRad). Blots were blocked with 5% BSA (bovine serum albumin, Sigma-Aldrich, L'Isle d'Abeau, France) in Tris-buffered saline containing 0.05% Tween-20 and then incubated overnight with the same buffer at 1% BSA with either anti-TF (1/1500) or anti-PSGL-1 antibodies (1/200), followed by incubation with a peroxidase-conjugated goat anti-mouse secondary antibody (1/50000, Sigma) and developed by a Amersham ECL Western blotting detection reagents and analysis system (GE Healthcare, France) using ImageQuant™ LAS 4000 camera (GE healthcare, France).

### Nanoparticle Tracking Analysis

The size distribution and concentration measurements were performed with a ZetaView® instrument (Zeta View® QUATT-NTA PMX-420, ParticleMetrix, Germany). Before measurements, samples were diluted to the appropriate concentration (between 2×10⁷ particles per ml and 1× 10⁸ particles per ml) in sterile PBS. Eleven positions were recorded for each sample. The acquisition was performed with the following parameters: laser wavelength: 488 nm, Sensitivity: 80, Shutter: 100.

### Zeta Potential Measurement

Zeta potential analyses were realized, after 1/100 dilution in NaCl 1 mM, using a NanoZS® apparatus equipped with DTS 1070 cell. All measurements were performed in triplicate at 25° C., with a dielectric constant of 78.5, a refractive index of 1.33, a viscosity of 0.8872 cP and a cell voltage of 150 V. The zeta potential was calculated from the electrophoretic mobility using the Smoluchowski equation.

### Exoview

Three independent batches of EV preparations for each condition were analyzed by ExoView R100 (NanoView Biosciences) following the manufacturer's protocol. Briefly, 35 μL of isolated EVs diluted in Exoview buffer to a concentration of 3E8part/ml (from NTA measurement) were incubated on the Exo View Tetraspanin Chip for human EVs and placed in a 24-well plate for 16 h at room temperature. The chips were washed three times then fixed for 10 min at room temperature with Exoview Cargo Kit Solution C. After three washes, chips were permeabilized with Exoview Cargo Kit solution D. Chips were then incubated 1 h with anti-CD63 AlexaFluor-488 (NanoView Biosciences), anti-calnexin AlexaFluor 647 (BD Biosciences) and anti-syntenin AlexaFluor 555 (NanoView Biosciences) antibodies, at a dilution of 400, 400 and 1000 respectively in the Exoview Blocking Solution. The chips were then washed and dried. Finally, the chips were imaged with the ExoScan 3.0 acquisition software and data was analyzed using Exo Viewer 3.0 with thresholds set to 400-20000 for the 488 nm channel, 300-20000 for the 555 nm channel and 710-20000 for the 647 nm channel according to the signal on the control isotype chips.

### Plasma Clotting Time

The effect of mEVs during clot formation was studied by monitoring the change in turbidity in human or mouse plasma using a microplate reader (Fluostar Optima, BMG Labtech). Calcium Chloride (25 mmol/L final concentration) was added to citrated plasma diluted 1:2 in HEPES buffer (10 mM HEPES, 150 mM NaCl, and 0.4% BSA, pH=7.4) to promote coagulation. Samples were incubated at 37° C. with mEVs at increasing doses and absorbance (405 nm) was monitored for 2 hours every 15 seconds at 37° C. When appropriate, anti-TF blocking antibody was also added to inhibit extrinsic pathway clot. Results are expressed as the time to achieve 75% maximal absorbance (75% Clotting time, CT). All experiments were performed in triplicate.

### Rotational Thromboelastography (ROTEM)

For thromboelastography assays, human fresh blood was drawn into 0.109 mol·L−1 trisodium citrate. Within 30 min, 300 μL of blood was added in EXTEM-S® or INTEM-S® reagents vial (-TEM®) in presence of increasing concentrations of mEVs (control, 2, 5 and 20×10 exp8) and clotting was monitored. When appropriated, blocking-TF antibody was added to prevent extrinsic pathway activation. Clotting time (CT, seconds), amplitude at 10 minutes (A10), clot formation time (CFT, seconds), maximal clot firmness (MCF, mm) and alpha angle (□ angle, °) were measured to characterize and quantify the coagulation activation, clot firmness and clot stability using rotational thromboelastography (ROTEM, TEM®). Blood of healthy donors (Etablissement Français du Sang, Ile de France) was collected and tested with the appropriate ethics prior approval as stated in the EFS/Inserm U1237 agreement #PLER-UPR/2018/017, ensuring that all donors gave a written informed consent, and providing anonymized samples.

### Thrombin Generation Assay

Thrombin generation measurements were performed pooled platelet free plasma with the ST Genesia® Thrombin Generation System (Stago, Asnières-sur-Seine, France). The reagent STG®-BleedScreen (STG-BLS) was selected since we wanted a test where thrombin generation was initiated by a mixture of procoagulant phospholipids and low picomolar level of human tissue factor (TF), balanced for sensitivity to procoagulant factor deficiencies while minimizing contact activation. Dose-response of mEVs (from 0-20×10 exp8 EVs/mL) was added and thrombin generation studied. When appropriated, anti-TF and Corn Tripsin inhibor (CTI) where added. A reference plasma and two quality controls for low and normal thrombin generation were tested for parameter normalization. The assay was triggered by dispensing a fluorogenic substrate and CaCl2. The following parameters were measured: lag time, peak height, time to peak, endogenous thrombin potential (ETP), ETP inhibition, and velocity index.

### Microfluidic Experiment

Microfluidic experiment was conducted using an ibidi pump system to control the blood flow through a microfluidic channel (μ-slide VI 0.4, ibidi) to reproduce hemostasis in vitro. First, the microfluidic channel was incubated with collagen from calf skin (Sigma) and left at room temperature for an hour. Whole blood collected on citrate from healthy volunteers was provided by Etablissement Français du Sang (EFS, Caen, France). Platelets of collected blood were stained using DiOC6 intracellular probe (1 μg/mL, Abcam). Blood was perfused through the microfluidic channel, reproducing an endothelial damage in the vasculature, at a shear rate of 300 s−1 according to previous study (ref). After extended washing of the blood, a preparation of mEVs (~10×10 exp9/mL EVs). When appropriate, mEVs solutions were pre-incubated 30 minutes with blocking anti-PSGL-1 (20 μg/mL) antibodies before injection in the chamber.

### Erythrocyte Hemolysis Assay

The erythrocyte toxicity of the mEVs was conducted as described by Mazzarino et al32 with minor modifications on human blood. Briefly, 1 mL of human fresh blood was drawn into 0.109 mol·L−1 trisodium citrate by venous puncture. Erythrocytes were purified by centrifugation at 2000 g for 5 min and washed three times in 10 mL of saline. Then, 50 μl of purified and washed erythrocytes were added to 950 μL of saline containing different concentrations of mEVs (control, 2, 5 and 20×10 exp8/mL final concentration) and incubated in 37 C under mild agitation. Then, erythrocytes were removed by centrifugation at 2000 g for 5 min. Then, the absorbance of the resulting supernatant was measured by UVvis and by spectrometer at 540 nm. Saline control solution (0.9% NaCl) and distilled water were used as negative (0% lysis) and positive (100% lysis) controls respectively. The hemolysis rate at 540 nm was calculated as follows: Hemolysis rate (%)=(As—Ac−/Ac+—Ac−)×100. Were As, Ac− and Ac+ represent the absorbance of the sample, Negative and positive controls respectively32. The experiments were performed in triplicate.

### Animals

All experiments were performed on 8 to 12-week-old male Swiss mice (Janvier, France). Animals were maintained under specific pathogen-free conditions at the Centre Universitaire de Ressources Biologiques (CURB, Basse-Normandie, France) and all had free access to food and tap water. Experiments were approved by the local ethical committee of Normandy (CENOMEXA, APAFIS #17834). For surgeries, mice were anesthetized with isoflurane 5% and maintained under anaesthesia with 2% isoflurane in a 70%/30% gas mixture (N2O/O2). The rectal temperature was maintained at 37.5° C. throughout using a feedback-regulated heating system. A catheter was inserted into the tail vein of mice for intravenous administration of mEVs.

### Tail Bleeding Assay

Animals were anesthetized with isofluorane a mixture of ketamine, xylazine and atropine (at 100, 10 and 1.2 mg/kg, respectively) and body weight (accurate to mg) was obtained. Animals were placed in prone position. A distal 2-mm segment of the tail was amputated with a scalpel. The tail was immediately immersed in a 1.5 mL Epperdorf tube containing isotonic saline pre-warmed in a water bath to 37° C. The position of the tail was vertical with the tip positioned about 2 cm below the body horizon. Each animal was monitored for 30 min even if bleeding ceased, to detect any re-bleeding. Bleeding time was determined using a stop clock. The experiment was terminated at the end of 30 min.

When appropriated, animals received i.o. injection of mEVs at 1 mg/kg before (1 minute) or after (1- or 15 minutes) tail amputation.

### IntraCranial Hemorrhage (ICH) Model

A unilateral striatal injection of collagenase type VII (0.1 U in 1 μL of saline) was performed after placing Swiss male mice (35 g) in a stereotaxic frame (coordinates: 0.5 mm anterior, 2.5 mm lateral, −3 mm ventral to the bregma). Solutions were injected using a glass micropipette to minimize hemorrhage-unrelated tissular damage. The needle was then removed, the craniotomy closed with surgical wax and the scalp sutured. Mice received an intra-peritoneal injection of buprenorphine (0.03 mg/kg) 20 minutes before and were anesthetized in 2% isoflurane in a mixture of O2/N2O 33%/67% with a rectal temperature maintained at 37° C.

mEVs Hemostatic Treatment

mEVs were diluted in saline and administered i.v. in 200 μl bolus (at 1 mg/kg~4×10 exp9 EVs, unless otherwise stated). Treatment was given 30 minutes after hemorrhagic stroke. Catheter incision was closed, and mice were allowed to recover.

When appropriate, mEVs solutions were pre-incubated 30 minutes with blocking anti-TF (20 μg/mL) or anti-PSGL-1 (20 μg/mL) antibodies before injection, as previously described.

### Enoxaparin Treatment

Enoxaparin (Sigma-Aldrich) was dissolved in saline and administered i.p. (2 mg/kg) after further dilution in saline (50%/50%). Treatment was given 10 minutes before hemorrhagic stroke.

### Warfarin Treatment

Warfarin (Tahor, Pfizer) was dissolved in distilled water and administered per os (2 mg/kg). Warfarin was given at 36 h before hemorrhagic stroke. Coagucheck INRange, a reader for the INR measurement as part of the monitoring of treatment with vitamin K antagonists was used before the surgery to confirm that animals were >1.5 INR index.

### Magnetic Resonance Imaging (MRI)

Experiments were carried out on a Pharmascan 7 T/12 cm system using surface coils (Bruker, Germany). T2-weighted images were acquired using a MSME sequence: TE/TR 51 ms/2500 ms with 70 μm*70 μm*500 μm spatial resolution. 3D T2*-weighted gradient echo imaging with flow compensation (GEFC, spatial resolution of 93 μm*70 μm*70 μm interpolated to an isotropic resolution of 70 μm) with TE/TR 12.6 ms/200 ms and a flip angle (FA) of 24° was performed to visualize MPIOs (acquisition time=15 min). All T2*-weighted images presented in this study are minimum intensity projections of 4 consecutive slices (yielding a Z resolution of 280 μm). Lesion sizes were quantified blinded to the experimental data on T2-weighted images and signal voids quantification on 3D T2*-weighted images using automatic Otsu tresholding in ImageJ software (v1.45r).

### Clinical Neurological Score

All mice were assessed at 4 h, 24 h using a 6-point Neuroscore scale (0=no apparent deficit; 1=slight deficit; 2=circling; 3=heavy circling; 4=no movement; 5=death) as described by Foerch and collaborators.

### Passive IntraCranial Hemorrhage (ICH) Model

A unilateral striatal injection of heterologous blood was performed after placing Swiss male mice (35 g) in a stereotaxic frame (coordinates: 0.5 mm anterior, 2.5 mm lateral, −3 mm ventral to the bregma). Heterologous blood was obtained after intracranial puncture in the torax of anesthetized mice. Blood was drawn into a sterile 26 Gauge needle, which was connected to an infusion system containing a 50 μL micro-syringe. The 26 Gauge was introduced into the right striatum. At a rate of 1 μL/min, 5 μL of blood was infused over 5 minutes followed by a waiting period of 10 minutes to allow clotting of the injected blood within the injection site. Mice received an intra-peritoneal injection of buprenorphine (0.03 mg/kg) 20 minutes before and were anesthetized in 2% isoflurane in a mixture of O2/N2O 33%/67% with a rectal temperature maintained at 37° C.

### Method for PSGL-1 ELISA

Lysed EVs samples were analyzed in order to quantify the concentration of PSGL-1 by ELISA. First monocyte cell-line (THP-1 cells) and human dermal microvascular endothelial cells (HMEC-1) were stimulated with different cytokines (either TNF (1-10 or 100 ng/mL), LPS (10 ng/mL) or PMA (10 ng/mL)) for 48 h in order to promote EVs production. Then, EVs were purified by serial centrifugations, as previously described, lysed with TNT (Tris-NaCl-Triton) buffer to measure total protein with BCA and finally used for human PSGL-1 ELISA analysis (Biotechne). HMEC-EVs were used as a negative control (no detection of PSGL-1)

### Statistical Analysis

Results are presented as the mean±SD. Statistical analyses were performed using Mann-Whitney's U-test. When more than two groups were compared, statistical analyses were performed using Kruskal-Wallis (for multiple comparisons) followed by post-hoc Mann-Whitney's U-test. When comparing two groups, a p-value <0.05 was considered significant (two sided).

### Results

Generation of Large-Amounts of Monocytic-Derived EVs (mEVs) in Bioreactors.

Our first objective was to produce mEVs in quantity large enough for in vivo studies. To this aim, monocytic cell-line THP-1 were grown in bioreactors and treated with TNF to generate mEVs (Data not shown). mEVs accumulated in supernatants from TNF-stimulated monocytes and were purified by sequential ultra-centrifugation as previously reported33. mEVs were labeled using a fixable-cell-plasma-membrane marker, immobilized on polydopamine coated micro-wells and detected by laser scanning confocal microscopy (LSCM) using an automated analysis procedure in ImageJ. There was a linear relationship between the number of surface-immobilized mEVs and their concentration (Data not shown). By LSCM, 80% of the apparent MP diameters ranged from 100 nm to 1.2 μm with a median size of 334 nm, which is similar to previously reported EVs sizes (Data not shown). The few detected EVs with apparent diameter >1 μm may correspond to aggregates of smaller EVs. Then we performed quantitative and morphological analyses by Nanoparticle tracking analysis (NTA using Zetasizer). Results showed that we were able to produce 4*1010 mEVs per batch, corresponding to ~400 μg of proteins. (Data not shown). According to NTA analysis, 95% of the mEVs population had a diameter below 500 nm, and the mean size was 200 nm (Data not shown). The discrepancies between LSCM and NTA results are expected given the differences in mEVs processing and measuring methods. Finally, tetraspanin markers (surface markers CD63, CD81 and CD9 and cytosolic Syntenin) were measured by Exoview (Data not shown). Results show that mEVs produced in bioreactors present the expected markers of extracellular vesicles, including low expression of Calnexin (used as a negative control to differentiate extracellular vesicles from exosomes, as proposed by ISEV recommendations) (Data not shown). Altogether, these results demonstrate that mEVs harboring classical markers of extracellular vesicles can be obtained in bioreactors.

Detection of mEVs Monocytic-Antigens Using Immunolabelling-LSCM and Immunoblot.

Then, we wanted to confirm that our isolated mEVs present monocytic surface markers, including CD14, Tissue factor (TF, that is known to be up-regulated after TNF stimulation), and P-Selectin Ligand-1 (PSGL-1). To this aim, we used fluorescent immunolabeling and LSCM. We immobilized the mEVs on polydopamine-coated micro-wells and performed high resolution LSCM with or without anti-CD14, anti-TF and anti-PSGL-1 primary antibodies coupled to fluorescently labelled secondary antibodies. No detergent was added to preserve MP membrane integrity and prevent antibody to reach intravesicular antigens. We were able to detect these three monocytic markers at the surface of mEVs (Data not shown). We confirmed these results by immunoblot of lysed mEVs. TNF-stimulated mEVs harbored high amounts of TF and PSGL-1 (Data not shown), as compared to controls (Data not shown). Therefore, we confirmed that our protocol allows production of mEVs harboring both TF and PSGL-1 on their surface.

mEVs Pro-Coagulant Effects are Mediated by the Activation of the Extrinsic Pathway In Vitro.

After having confirmed that the mEVs produced in bioreactors present the expected morphological characteristics and surface markers, we wanted to investigate their hemostatic potential. To that aim, we performed plasma (EVs-depleted) clotting assay using increasing concentrations of purified mEVs. mEVs present high pro-coagulant activity reducing the clotting time in a dose-dependent manner. The higher dose (160*106 mEVs) was able to reduce the clotting time by more than 53% compared to control condition (0 EVs) (Data not shown). To determine if the hemostatic potential was dependent on the activation of FVIIa by TF on the surface of the mEVs, we used a blocking anti-TF antibody (20 μg/mL). The addition of anti-TF significantly reduced the pro-coagulant effect of mEVs. To confirm these results and determine whether mEVs activate mainly the extrinsic or the intrinsic coagulation pathways, we repeated the clotting experiments using whole blood and thromboelastrometry (ROTEM). mEVs had an additive effect and reduced the clotting time in a dose-response manner when the coagulation was triggered with kaolin (INTEM-S), which activates the intrinsic pathway (Data not shown). In contrast, no effect was observed when coagulation was triggered by an extrinsic pathway activator (EXTEM-S) (Data not shown). In other words, the pro-coagulant effect of mEVs is masked in the presence of a full activation of the extrinsic pathway. This result suggests that most of the pro-coagulant effects of mEVs is dependent on the activation of the extrinsic pathway. In line with these results, their pro-coagulant effects in ROTEM experiments were blocked in the presence of an anti-TF antibody (20 μg/mL) (Data not shown).

To further study the effects of mEVs on the coagulation cascade, thrombin generation (TG) activity capacity was measured in the presence of mEVs using ST Genesia TG System (Stago) and STGR-BleedScreen reagent, that uses low picomolar level of TF to trigger coagulation. Dose-response of mEVs (from 0-20*108 EVs/mL) showed that the time to peak shortened and the thrombin generated (peak weight) was higher in a EVs dose-dependent manner compared to control (0 EVs) and reference plasma (Data not shown). Inhibition of extrinsic pathway using CTI resulted in little or no changes to the TG profile, further indicating that the extrinsic pathway is involved in the procoagulant effect of mEVs (Data not shown). Moreover, less thrombin was generated in the presence of an anti-TF antibody (Data not shown). Besides, the biocompatibility of mEVs was also briefly investigated in vitro. Hemolysis was unaffected by mEVs administration using human blood (Data not shown). All these results support that purified mEVs trigger coagulation mainly by the extrinsic coagulation pathway and do not induce hemolysis.

Intravenous Injection of mEVs Reduces Tail Bleeding Time In Vivo.

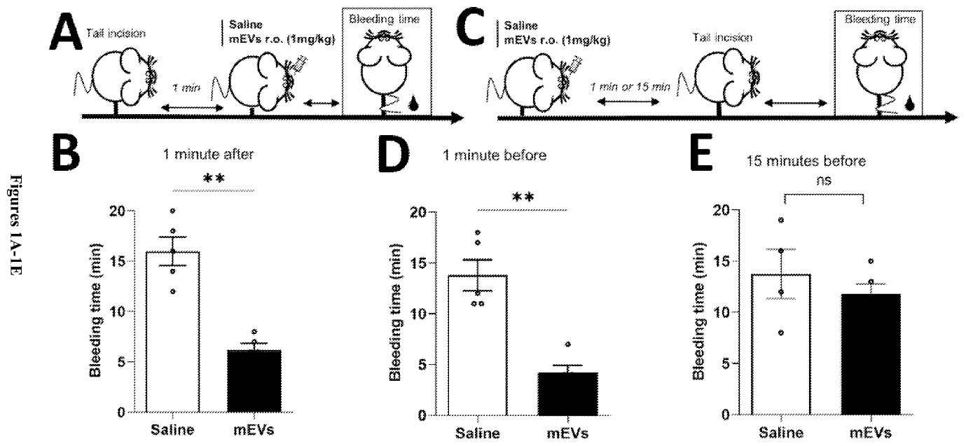

Then, we wanted to study the hemostatic effects of mEVs in vivo. To that aim, we used the mouse tail bleeding model, a well-established model to study hemostasis. First, mEVs (1 mg/kg) were injected intra-orbital 1 minute after the tail incision. mEVs administration reduced the bleeding time and blood loss by 55%, compared to the control group (FIGS. 1A and B). A similar trend in the reduction of bleeding time and blood loss was observed when mEVs were injected 1 minute before incision (reduction of the bleeding time by 68% compared to controls, FIGS. 1C and D). In contrast, intra-orbital mEVs injection did not significantly reduce the bleeding time or blood loss when administered 15 minutes before tail incision (FIG. 1E). This last result suggests that the hemostatic effect is transient, probably because of a short plasmatic half-life of mEVs. Overall, these results demonstrate that intra-orbital injection of exogenous mEVs reduces bleeding time in mice.

Dose-Response Study of mEVs in a Model of Intracranial Hemorrhage (ICH) in Mice.

We further evaluated the ability of mEVs to promote hemostasis in an ICH model in mice, induced by the intrastriatal injection of collagenase VII (Data not shown). In this model, mEVs infusion was performed 30 minutes after the onset of ICH. We first performed a pilot dose-response study of mEVs (from 0 to 1 mg/kg) (Data not shown). Hematoma volume was quantified by MRI at 24 h and neurological deficits were measured at 4 h and 24 h post-ICH (Data not shown). Only mEVs at the highest dose (1 mg/kg) significantly reduced ICH volume (Data not shown) and improved neurological score at 24 h (Data not shown) compared to control mice (n=5/group). Moreover, there was a dose-response relationship between mEVs and ICH volume (p for trend <0.05).

mEVs with Hemostatic Potential Improve Stroke Outcome in Mild and Severe Hemorrhagic Strokes.

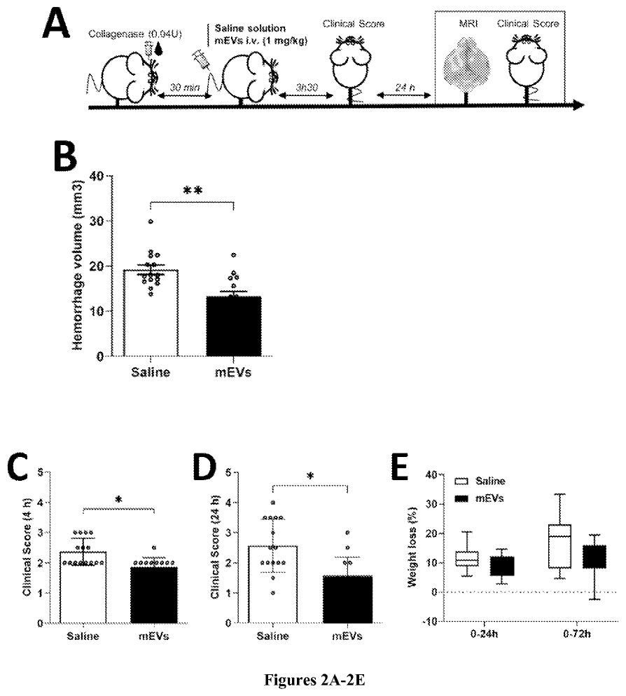

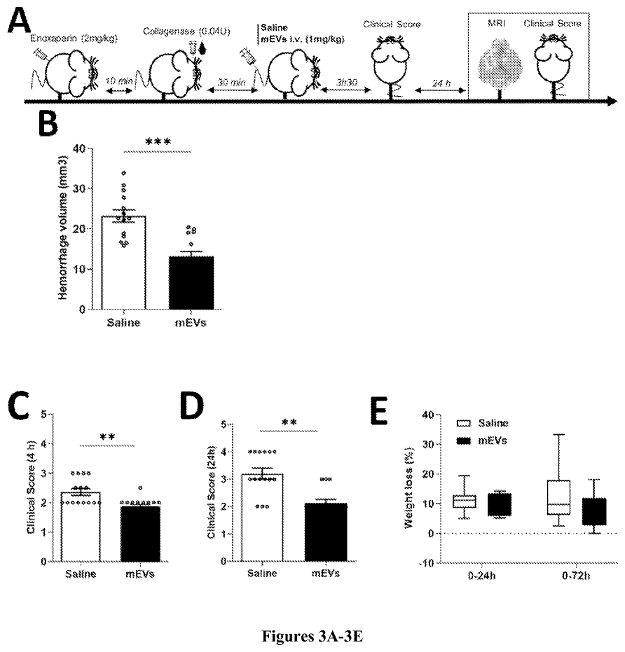

Thereafter, we wanted to confirm the results of our pilot dose-response experiment using a larger number of animals per group. We selected the 1 mg/kg mEVs dose according to our previous results (FIG. 2A). In a confirmatory experiment involving 15 mice per group, mEVs administration at 1 mg/kg, 30 minutes after ICH onset, reduced the ICH volume by 43% (Data not shown and FIG. 2B) and improved neurological score at 4 h and 24 h compared to control mice (FIGS. 2C-2E, p<0.01). Then, we wanted to confirm if this beneficial effect was also present in a more severe model of ICH. To mimic a frequent clinical situation where ICH occurs in anticoagulated patients, we pre-treated the mice with either enoxaparin (2 mg/kg i.p., 10 minutes before collagenase injection) or warfarin (2 mg/kg per os, 36 hours before collagenase injection). Enoxaparin pre-treatment led to larger hematoma volumes (from ~18 to ~24 mm3). Intravenous injection of mEVs also presented beneficial effect in enoxaparin treated mice (FIG. 3A), reducing by 40% the hematoma volume (FIG. 3B) and improving the clinical score at 24 h compared to control mice (FIGS. 3C-E). Mice pre-treated with warfarin presented low survival rates in the ICH model. The averaged survival rate increased from 12.5% to 55% after intravenous injection of mEVs compared to control mice (p<0.05, Data not shown).

The Beneficial Effects of mEVs in ICH are Mediated by Both TF and PSGL-1.

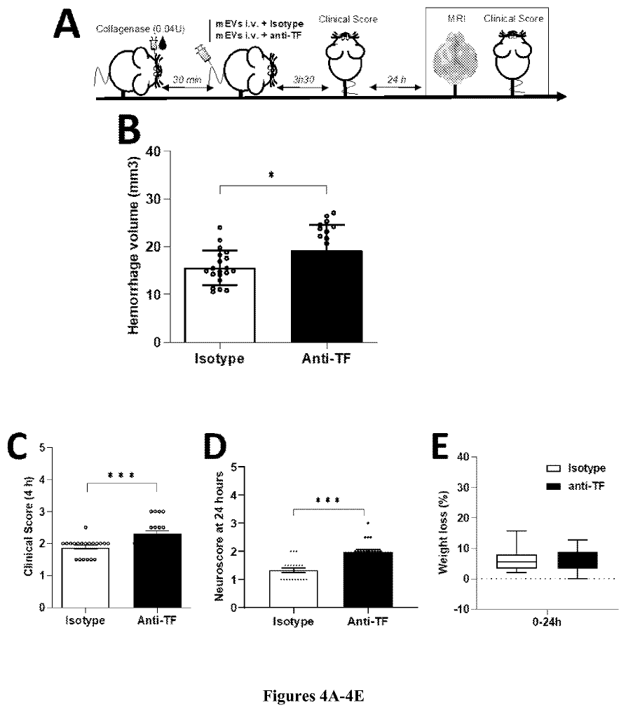

To gain mechanistic insights on the beneficial effect of mEVs in ICH, we studied the role of two key proteins harbored at the surface of mEVs: TF and PSGL-1. The role of mEVs expressing TF in clot propagation has been established in vivo in previous studies, but the ability of exogenous mEVs to localize to sites of injury and locally promote hemostasis remains unknown. To examine this hypothesis in vivo, mEVs were preincubated with either TF-blocking antibodies or control isotype antibodies and tested in the collagenase-induced ICH model (FIG. 4A). Blocked-TF-mEVs showed no beneficial effect as compared to control mEVs in terms of hematoma volume (Data not shown and FIG. 4B) and clinical score (FIGS. 4C-E). This result supports the key role of TF in the beneficial effect of mEVs.

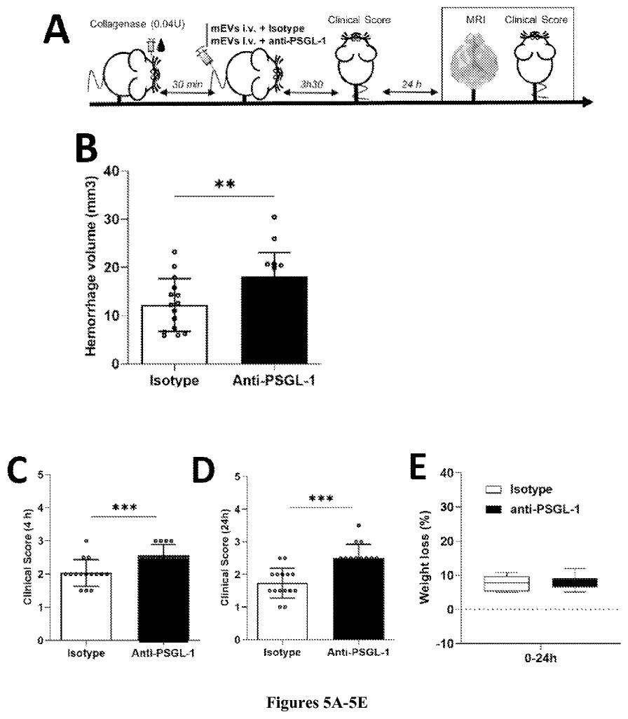

Previous work demonstrated that incorporation of monocyte-derived, TF-positive EVs into a thrombus is mediated by the interaction of PSGL-1 on the surface of the EVs with P-selectin on the surface of activated platelets and endothelial cells. Thus, we wanted to confirm that the beneficial effect of mEVs administration in ICH was dependent on PSGL-1 binding on P-Selectin. To examine this hypothesis in vivo, mEVs were preincubated with either PSGL-1-blocking antibodies or control isotype antibodies and tested in the collagenase-induced ICH model (FIG. 5A and Data not shown). As observed in FIG. 5B, PSGL-1 blocked mEVs failed to reduce ICH volume or to improve the clinical score at 24 h (FIGS. 5C-5E). Therefore, these results support that both TF and PSGL-1 at the surface of the mEVs are critical for their beneficial effect.

Besides, we confirm the specificity of fixation of EVs to platelet-forming microthrombi, we used microfluidics as previously described. EVs were labelled with orange cell-membrane dye on the surface to be observed in fluorescence. Our result shows that EVs are capable to bind on the in vitro-created platelet microthrombi formed into the microfluidic chamber (Data not shown) when injected with human blood. For a better understanding of the mechanistic, we preincubated EVs with anti-PSGL-1 blocking antibody. When injected blocked-EVs in whole human blood, the number of EVs attached to microthrombi was highly reduced compared to control EVs, showing that PSGL-1 at their surface impact EVs fixation.

mEVs do not Worsen ICH Outcome in an ICH Model Involving Direct Intracranial Heterologous Blood Injection.

One of the risks associated with the use of hemostatic agents at the acute phase of ICH is the potential aggravation of ischemia and oedema occurring at the periphery of the hematoma. Thus, to isolate the putative deleterious effects of mEVs from their beneficial effects, we used an ICH model that consists in the direct injection of heterologous blood. In this model devoid of active bleeding, we treated the mice with mEVs (1 mg/kg) and assessed edema and lesion size at 24 hours by T2 and diffusion weighted images. mEVs neither aggravated edema nor lesion size compared to control, supporting a favorable safety profile (Data not shown).

### Quantification of Human PSGL-1 Levels in Different EVs Populations by ELISA

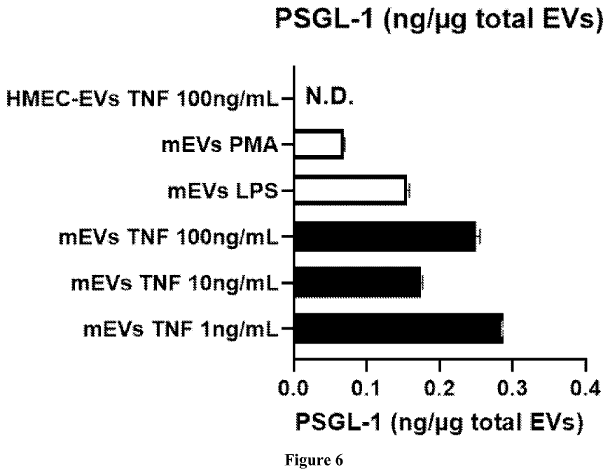

Finally, we compared the total amount of PSGL-1 in EVs produced with different pro-inflammatory stimuli (cytokines) with ELISA. We found a high concentration of PSGL-1 superior to 0.1 ng PSGL-1/μg monocyte-EVs proteins, more precisely superior to 0.2 ng PSGL-1/μg monocyte-EVs proteins at the surface of the mEVs (FIG. 6).

### REFERENCES

Throughout this application, various references describe the state of the art to which this invention pertains. The disclosures of these references are hereby incorporated by reference into the present disclosure.
1. Garg R, Biller J. Recent advances in spontaneous intracerebral hemorrhage. F1000Res. 2019; 8
2. Cordonnier C, Demchuk A, Ziai W, Anderson C S. Intracerebral haemorrhage: Current approaches to acute management. Lancet. 2018; 392:1257-1268
3. Qureshi A I, Tuhrim S, Broderick J P, Batjer H H, Hondo H, Hanley D F. Spontaneous intracerebral hemorrhage. N. Engl. J. Med. 2001; 344:1450-1460
4. Broderick J P, Adams H P, Jr., Barsan W, Feinberg W, Feldmann E, Grotta J, et al. Guidelines for the management of spontaneous intracerebral hemorrhage: A statement for healthcare professionals from a special writing group of the stroke council, american heart association. Stroke. 1999; 30:905-915
5. Anderson C S, Heeley E, Huang Y, Wang J, Stapf C, Delcourt C, et al. Rapid blood-pressure lowering in patients with acute intracerebral hemorrhage. N. Engl. J. Med. 2013; 368:2355-2365
6. Qureshi A I, Palesch Y Y. Antihypertensive treatment of acute cerebral hemorrhage (atach) ii: Design, methods, and rationale. Neurocrit. Care. 2011; 15:559-576
7. Carhuapoma J R, Barrett R J, Keyl P M, Hanley D F, Johnson R R. Stereotactic aspiration-thrombolysis of intracerebral hemorrhage and its impact on perihematoma brain edema. Neurocrit. Care. 2008; 8:322-329
8. Hanley D F, Thompson R E, Rosenblum M, Yenokyan G, Lane K, McBee N, et al. Efficacy and safety of minimally invasive surgery with thrombolysis in intracerebral haemorrhage evacuation (mistie iii): A randomised, controlled, open-label, blinded endpoint phase 3 trial. Lancet. 2019; 393:1021-1032
9. Goldstein J N, Fazen L E, Snider R, Schwab K, Greenberg S M, Smith E E, et al. Contrast extravasation on ct angiography predicts hematoma expansion in intracerebral hemorrhage. Neurology. 2007; 68:889-894
10. Cordonnier C, Tymianski M. Mistie iii. Stroke. 2019; 50:1634-1635
11. Alvarez-Sabín J, Delgado P, Abilleira S, Molina C A, Arenillas J, Ribó M, et al. Temporal profile of matrix metalloproteinases and their inhibitors after spontaneous intracerebral hemorrhage: Relationship to clinical and radiological outcome. Stroke. 2004; 35:1316-1322
12. Yang G Y, Betz A L, Chenevert T L, Brunberg J A, Hoff J T. Experimental intracerebral hemorrhage: Relationship between brain edema, blood flow, and blood-brain barrier permeability in rats. J. Neurosurg. 1994; 81:93-102
13. Brott T, Broderick J, Kothari R, Barsan W, Tomsick T, Sauerbeck L, et al. Early hemorrhage growth in patients with intracerebral hemorrhage. Stroke. 1997; 28:1-5
14. Li Q, Zhang G, Xiong X, Wang X C, Yang W S, Li K W, et al. Black hole sign: Novel imaging marker that predicts hematoma growth in patients with intracerebral hemorrhage. Stroke. 2016; 47:1777-1781
15. Davis S M, Broderick J, Hennerici M, Brun N C, Diringer M N, Mayer S A, et al. Hematoma growth is a determinant of mortality and poor outcome after intracerebral hemorrhage. Neurology. 2006; 66:1175-1181
16. Li Q, Huang Y J, Zhang G, Lv F J, Wei X, Dong M X, et al. Intraventricular hemorrhage and early hematoma expansion in patients with intracerebral hemorrhage. Sci. Rep. 2015; 5:11357
17. Tuhrim S, Dambrosia J M, Price T R, Mohr J P, Wolf P A, Hier D B, et al. Intracerebral hemorrhage: External validation and extension of a model for prediction of 30-day survival. Ann. Neurol. 1991; 29:658-663
18. Portenoy R K, Lipton R B, Berger A R, Lesser M L, Lantos G. Intracerebral haemorrhage: A model for the prediction of outcome. J. Neurol. Neurosurg. Psychiatry. 1987; 50:976-979
19. Daverat P, Castel J P, Dartigues J F, Orgogozo J M. Death and functional outcome after spontaneous intracerebral hemorrhage. A prospective study of 166 cases using multivariate analysis. Stroke. 1991; 22:1-6
20. Broderick J P, Brott T G, Duldner J E, Tomsick T, Huster G. Volume of intracerebral hemorrhage. A powerful and easy-to-use predictor of 30-day mortality. Stroke. 1993; 24:987-993
21 Gulati D, Dua D, Torbey M T. Hemostasis in intracranial hemorrhage. Front. Neurol. 2017; 8:80
22. Mackman N, Tilley R E, Key N S. Role of the extrinsic pathway of blood coagulation in hemostasis and thrombosis. Arterioscler. Thromb. Vasc. Biol. 2007; 27:1687-1693
23. Mayer S A, Brun N C, Begtrup K, Broderick J, Davis S, Diringer M N, et al. Efficacy and safety of recombinant activated factor vii for acute intracerebral hemorrhage. N. Engl. J. Med. 2008; 358:2127-2137
24. Mayer S A, Brun N C, Broderick J, Davis S M, Diringer M N, Skolnick B E, et al. Recombinant activated factor vii for acute intracerebral hemorrhage: Us phase iia trial. Neurocrit. Care. 2006; 4:206-214
25. Yáñez-Mó M, Siljander P R, Andreu Z, Zavec A B, Borras F E, Buzas E I, et al. Biological properties of extracellular vesicles and their physiological functions. J Extracell Vesicles. 2015; 4:27066
26. Théry C, Amigorena S, Raposo G, Clayton A. Isolation and characterization of exosomes from cell culture supernatants and biological fluids. Curr. Protoc. Cell Biol. 2006; Chapter 3: Unit 3.22
27. Zubairova L D, Nabiullina R M, Nagaswami C, Zuev Y F, Mustafin I G, Litvinov R I, et al. Circulating microparticles alter formation, structure, and properties of fibrin clots. Sci. Rep. 2015; 5:17611
28. Owens A P, 3rd, Mackman N. Microparticles in hemostasis and thrombosis. Circ. Res. 2011; 108:1284-1297
29. Mege D, Mezouar S, Dignat-George F, Panicot-Dubois L, Dubois C. Microparticles and cancer thrombosis in animal models. Thromb. Res. 2016; 140 Suppl 1:S21-26
30. Thomas G M, Panicot-Dubois L, Lacroix R, Dignat-George F, Lombardo D, Dubois C. Cancer cell-derived microparticles bearing p-selectin glycoprotein ligand 1 accelerate thrombus formation in vivo. J. Exp. Med. 2009; 206:1913-1927
31. Falati S, Liu Q, Gross P, Merrill-Skoloff G, Chou J, Vandendries E, et al. Accumulation of tissue factor into developing thrombi in vivo is dependent upon microparticle p-selectin glycoprotein ligand 1 and platelet p-selectin. J. Exp. Med. 2003; 197:1585-1598
32. Mazzarino L, Loch-Neckel G, Dos Santos Bubniak L, Ourique F, Otsuka I, Halila S, et al. Nanoparticles made from xyloglucan-block-polycaprolactone copolymers: Safety assessment for drug delivery. Toxicol. Sci. 2015; 147:104-115
33. Briens A, Gauberti M, Parcq J, Montaner J, Vivien D, Martinez de Lizarrondo S. Nano-zymography using laser-scanning confocal microscopy unmasks proteolytic activity of cell-derived microparticles. Theranostics. 2016; 6:610-626
34. Piffoux M, Volatron J, Cherukula K, Aubertin K, Wilhelm C, Silva A K A, et al. Engineering and loading therapeutic extracellular vesicles for clinical translation: A data reporting frame for comparability. Adv Drug Deliv Rev. 2021; 178:113972
35. Silva A K A, Morille M, Piffoux M, Arumugam S, Mauduit P, Larghero J, et al. Development of extracellular vesicle-based medicinal products: A position paper of the group “extracellular vesicle translation to clinical perspectives-evolve france”. Adv Drug Deliv Rev. 2021; 179:114001
36. Herrmann I K, Wood M J A, Fuhrmann G. Extracellular vesicles as a next-generation drug delivery platform. Nat Nanotechnol. 2021; 16:748-759
37. Gladstone D J, Aviv R I, Demchuk A M, Hill M D, Thorpe K E, Khoury J C, et al. Effect of recombinant activated coagulation factor vii on hemorrhage expansion among patients with spot sign-positive acute intracerebral hemorrhage: The spotlight and stop-it randomized clinical trials. JAMA neurology. 2019; 76:1493-1501
38. S ELA, Mäger I, Breakefield X O, Wood M J. Extracellular vesicles: Biology and emerging therapeutic opportunities. Nat Rev Drug Discov. 2013; 12:347-357
39. Kuravi S J, Yates C M, Foster M, Harrison P, Hazeldine J, Hampson P, et al. Changes in the pattern of plasma extracellular vesicles after severe trauma. PLOS One. 2017; 12: e0183640
40. Thangaraju K, Neerukonda S N, Katneni U, Buehler P W. Extracellular vesicles from red blood cells and their evolving roles in health, coagulopathy and therapy. International journal of molecular sciences. 2020; 22:153
41. Del Conde I, Shrimpton C N, Thiagarajan P, López J A. Tissue-factor-bearing microvesicles arise from lipid rafts and fuse with activated platelets to initiate coagulation. Blood. 2005; 106:1604-1611

1. Monocyte extracellular vesicles (mEVs) functionalized with tissue factor (TF) and P-Selectin Glycoprotein Ligand 1 (PSGL-1).

2. The monocyte extracellular vesicles according to claim 1 having a concentration of PSGL-1 superior to 0.1 ng PSGL-1/μg monocyte-EV proteins.

3. The monocyte extracellular vesicles according to claim 1 having a concentration of PSGL-1 superior to 0.2 ng PSGL-1/μg monocyte-EV proteins.

4. The monocyte extracellular vesicles according to claim 1, wherein a concentration of PSGL-1 is measured by ELISA.

5. The monocyte extracellular vesicles according to claim 1, having a diameter of 300 nm.

6. The monocyte extracellular vesicles according to claim 1, which are exogenous monocyte extracellular vesicles.

7. (canceled)

8. A method of producing the monocyte extracellular vesicles according to claim 1, wherein the monocyte extracellular vesicles are produced in bioreactors after pro-inflammatory cytokine stimulation or shear stress.

9. A method for treating intracerebral hemorrhage in a subject in need thereof comprising administering to the subject a therapeutically effective amount of the exogenous monocyte extracellular vesicles according to claim 6.

10. The method of claim 9, wherein the intracerebral hemorrhage is stroke.

11. (canceled)

12. The method according to claim 9, wherein the monocyte extracellular vesicles are administered in combination with one or more of a fibrinolysis inhibitor, an antagonist of anticoagulant treatments, an inhibitor of a direct oral anticoagulant, or a coagulation factor.

13. The method according to claim 12, wherein the fibrinolysis inhibitor is tranexamic acid.

14. The method according to claim 12, wherein the antagonist of anticoagulant treatments is protamine or heparin.

15. The method according to claim 12, wherein the coagulation factor is a prothrombin complex concentrate or vitamin K.
